SEQUENCE LISTING (28 sequences)

SEQ ID NO: 1 - DNA, 1506 bp, synthetic construct
  source 1..1506: mol_type = other DNA; note = An optimized nucleotide sequence for tHMG1S gene
atgctgaccaacaagaccgtcatctctggttccaaggtcaagtccttgtcctctgcacagtcctcctcttctggtccatcctcctcttctgaggaagacgactctcgtgatattgagtccttggacaagaagatccgtccactcgaagaactggaagccttgttgtcctctggtaacaccaagcaactgaagaacaaggaggtcgctgcattggtcatccacggtaagttgccactgtacgcactggagaagaagttgggtgacactactcgtgctgttgctgtccgtcgtaaggcactgtccatcttggctgaagcacctgtcttggcatctgatcgtttgccatacaagaactacgactacgaccgtgtctttggtgcctgttgtgagaacgtcatcggttacatgccactgcctgttggtgtcatcggtccactggtcatcgatggtacttcctaccacatccctatggccactactgagggttgcctggtcgcatctgcaatgcgtggttgcaaggcaatcaacgctggtggtggtgcaaccactgtcttgaccaaggatggtatgactcgtggacctgttgttcgtttcccaaccctgaaacgttctggtgcctgcaaaatctggctggactccgaagagggtcagaacgccatcaagaaggcattcaactccacctctcgtttcgcacgtctgcaacacattcagacctgtctcgctggtgacttgctcttcatgcgttttcgtaccactactggtgatgcaatgggtatgaacatgatctctaagggtgtcgagtactccttgaagcagatggtcgaagagtatggttgggaagacatggaggttgtctccgtttctggtaactactgcaccgacaagaagcctgctgccatcaactggatcgaaggtcgtggtaagtctgtcgtcgctgaagccaccattcctggtgatgttgttcgtaaggtcttgaagtctgacgtctctgcactggttgaactgaacattgccaagaacctggttggttctgcaatggctggttctgttggtggtttcaacgcacatgctgccaacttggtcactgctgtcttcttggcactgggtcaagatcctgcacagaacgtcgagtcttccaactgcatcaccctgatgaaggaagttgacggtgacttgcgtatctctgtctccatgccatccatcgaagttggtactattggtggtggtactgttctggaaccacaaggtgcaatgctggacttgttgggtgttcgtggtccacatgcaactgctcctggtactaacgcacgtcaactggcacgtatcgttgcatgtgctgtcctggctggtgagttgtccttgtgtgctgcactggctgctggtcatctggttcaatcccacatgactcacaatcgtaaacctgctgaaccaaccaaacctaacaacctggatgccactgacatcaaccgtctgaaggatggttccgtcacctgcatcaaatcctaa

SEQ ID NO: 2 - DNA, 1203 bp, synthetic construct
  source 1..1203: mol_type = other DNA; note = An optimized nucleotide sequence for HpPSY1S gene
atggctactccacttcctaccaagagaacccactccatcgtctctgttccacctgctatgctccatcagtctcttcctggtagatgcagaccacactcctccagatgtcctgttgctatctctgctaccctcgttggacctgaccctagatggtccatcgcatcctctcaggttgtccctaagcagccacagctcaagggaaaggacgtcgaggaggcagcaatgtggagatgcatcgacctccacagaagacttcctaacggaggtgcaccacagcaagcatccagatggactccagctactcttgaggaggcataccagagatgtggtcaagtcacctctgagttcgcaaagaccttctaccttggtactcagctcatgactccaatccaggcaaagtccatctgggctatctacgtctggtgtagaagaaccgacgagttggttgatggtccaaacgctaacaagatcactcctaaggcactcgacagatgggaggagagacttgaggctaccttcgctggaagaccatacgacgtcctcgatgcagcactctccgacaccatctctaagttcccaatggacatccaaccattcaaggacatgatcgaaggaatgagaatggacttgcacaagaccagataccagaccttcgacgaactctacgagtactgctacagagttgctggtactgttggactcatgactatgcctgttatgggtatcgatccatcttacaagggaccaatggatgtcgtctacaaggcagctcttgcacttggtactgctaaccagctcaccaacatcttgagagatgtcggtgaggatgctagagagagaaacagaatctaccttccaatggaggacctccagcagttcggattgaccgagcaggacgtccttggagcagtccacgttccatctcagggtaaggtctctgagaagtggagagcattcatgaagttccagatcgctagagctagacagtgcttcgcagatgctgagtctggtgttgaccagttggaggctaaggctagatggcctgtctggtctgctctcatcctctacagacagatcctcgacgctatcgagaagaacgactacgacaacttctcccagagagcttacgtctccaaggctaagaagatggcttctcttccactcgctctcaccagagcactccttccacagcacagaggttaa

SEQ ID NO: 3 - DNA, 1479 bp, synthetic construct
  source 1..1479: mol_type = other DNA; note = An optimized nucleotide sequence for PacrtIS gene
atgaagccaactactgtcatcggtgctggtttcggtggtctggctctggctatccgtctgcaagctgctggtatcccagtcctgctgctggaacaacgtgacaagccaggtggtcgtgcttacgtctacgaggaccagggtttcactttcgacgctggtccaactgtcatcactgacccatccgctatcgaggagctgttcgctctggctggtaagcagctgaaggagtacgtcgagctgctgccagtcactccattctaccgtctgtgctgggaatctggtaaggtcttcaactacgacaacgaccagactcgtctggaagctcagatccagcagttcaacccacgtgacgtcgaaggttatcgtcagttcctggactactcccgtgctgtcttcaaggaaggttatctgaagctcggtactgtcccattcctgtccttccgtgacatgctgcgtgctgctccacagctggctaagctccaggcttggcgttctgtctactccaaggtcgcttcctacatcgaagacgaacacctgcgtcaggctttctccttccactccctgctggtcggtggtaatccattcgctacttcctccatctacactctgatccacgctctggaacgtgaatggggtgtctggttcccacgtggtggtactggtgctctggtccagggtatgatcaagctgtttcaggacctgggtggtgaagtcgtcctgaacgctcgtgtctcccacatggaaactactggtaacaagatcgaggctgtccacctggaggacggtcgtcgtttcctgactcaggctgtcgcttccaacgctgacgtcgttcacacttaccgtgatctgctgtctcagcatccagctgctgtcaagcagtccaacaagctgcaaactaagcgtatgtccaactctctgttcgtcctgtacttcggtctgaaccaccatcacgaccagctggctcaccacactgtctgcttcggtccacgttaccgtgagctgatcgacgaaatcttcaaccacgacggtctggctgaggacttctctctgtacctgcacgctccatgcgtcactgactcctctctggctccagaaggttgtggttcctactacgttctggctccagtcccacacctgggtactgctaacctggactggactgtcgaaggtccaaagctgcgtgaccgtatcttcgcttacctggaacagcactacatgccaggtctgcgttctcagctggtcactcaccgtatgttcactccattcgacttccgtgatcagctgaacgcttaccacggttccgctttctccgtcgaaccagtcctgactcagtctgcttggttccgtccacacaaccgtgacaagactatcactaacctgtacctggtcggtgctggtactcatccaggtgctggtatcccaggtgtcatcggttccgctaaggctactgctggtctgatgctggaggacctgatctaa

SEQ ID NO: 4 - DNA, 528 bp, synthetic construct
  source 1..528: mol_type = other DNA; note = An optimized nucleotide sequence for PscrtZS gene
atgttgtggatttggaatgcactgatcgtcttcgttactgttgttggcatggaagttgttgctgcactggcacacaagtacatcatgcatggctggggttggggttggcatctgtcacatcatgaaccacgtaagggtgcattcgaagtgaacgacctgtatgctgtcgtgttcgcaatcgtctccatcgcactgatctacttcggttcaactggtatctggccactgcaatggatcggtgctggcatgactgcctacggtctcctgtacttcatggtgcatgatggtctggttcatcaacgttggccattccgttacatcccacgtaagggttatctgaagcgtctgtacatggcacaccgtatgcatcatgctgttcgtggcaaggagggttgcgtctccttcggtttcctgtatgcaccaccactctccaagctccaggcaactctgcgtgaacgtcatgctgctcgttctggtgctgcacgtgatgagcaggatggtgtggatacctcctcatctggcaagtaa

SEQ ID NO: 5 - DNA, 1689 bp, synthetic construct
  source 1..1689: mol_type = other DNA; note = An optimized nucleotide sequence for CsCCD2S gene
atggccaacaaggaggaggccgagaagaggaagaagaagcctaagcctctcaaggttctcatcactaaggttgaccctaagcctaggaagggcatggcctccgtggctgtggacctgctggagaaggccttcgtgtacctgctgtccggcaactccgctgccgacaggtcctcctcctccggcaggaggaggaggaaggagcactactacctgtccggcaactacgctcctgtgggccacgagactcctccgtccgaccacctgccgatccacggctccctgcctgagtgcctgaacggcgtgttcctgagggtcggtccgaacccgaagttcgcacctgtcgctggctacaactgggtggacggcgacggcatgatccacggcctgaggatcaaggacggcaaggccacctacctgtccaggtacatcaagacctccaggttcaagcaggaggagtacttcggcagggccaagttcatgaagatcggcgacttgaggggtctgctgggcttcttcaccatcctgatcctcgtgctgaggaccaccctgaaggtgatcgacatctcctacggcaggggcaccggtaacaccgcactggtgtaccacaacggcctgctgctggcactgtccgaggaggacaagccgtacgtcgtgaaggtgctggaggacggcgacctccagaccttgggcatcctggactacgacaagaagctgtcccacccgttcactgcccacccgaagatcgacccgctgaccgacgagatgttcaccttcggctactccatctctccgccgtacctgacctacagggtgatctccaaggacggtgtgatgcaggacccggtgcaaatctccatcacctccccgaccatcatgcacgacttcgccatcaccgagaactacgccatcttcatggacctgccgctgtacttccagcctgaggagatggtgaagggcaagttcgtgtcctccttccacccgaccaagagggctaggatcggcgtcctgccgaggtacgccaaggacgagcacccgatcaggtggttcgacttgccgagctgcttcatgacccacaacgccaacgcctgggaggagaacgacgaggtggtgctgttcacttgcaggctggagtctcctgacctggacatgctctccggtccggctgaggaggagatcggcaactccaagtccgagttgtacgagatgaggttcaacctgaagaccggcatcacctcccagaagcagctgtccgtcccgtccgtggacttcccgaggatcaaccagtcctacaccggcaggaagcagcagtacgtgtactgcactctgggcaacaccaagatcaagggcatcgtgaagttcgacttgcagatcgagcctgaggctggcaagaccatgttggaggtcggtggcaacgtccagggcatcttcgagctgggtccgaggaggtacggctccgaggccatcttcgtgccgtgccagcctggcatcaagtccgacgaggacgacggctacctgatcttcttcgtccacgacgagaacaacggcaagtccgaggtgaacgtgatcgacgccaagaccatgtccgctgagcctgtcgctgtcgtcgagctgccgtccagggtcccgtacggcttccacgcactgttcttgaacgaggaggagttgcagaagcaccaggctgagacctga

SEQ ID NO: 6 - DNA, 1602 bp, synthetic construct
  source 1..1602: mol_type = other DNA; note = An optimized nucleotide sequence for NcALD8S gene
atggctgcatccaaggttgagatcgcaccattcgaagtgactccactggatgctatccctgctgtgtgctcaactgcacgtgcaaccttcgcatcacacaagaccaagaacctccagtggcgtctggttcagctgcgtaagctgtactgggcactggatgacttcaaggcatcactcatggctgcactccagcaggatcttcgtaagggtggctacgagtctgacttcactgaagtggactgggtcaagaacgactgcttgcacatgatcaacaacctggagacctttgccaagactgagaagctcaaggacctgcctgtgacttactccatgatgaacttccgtgtcaagaaggagccactgggtactgttctcatcatcggaccatacaacttcccaatccagctggttcttgcaccactggttggtgccattggtgctggctgtactgctgtcatcaagccatctgagttgactcctgcctgcgctatggccatgaaggagatgatcgagtcacgtctggaccgtgacgcattcgctgttgtgaacggtggtgttcctgagactaacgcactgatggaggagaagtgggacaagatcatgttcactggctctgcacaggttggctccatcatcgcacgtaaggctgctgagactctgactcctgtgtgcctggaacttggtggacgtaaccctgccttcgtgaccaagaaggccaaccttgcacttgctgcacgtcgtctgatgtggggtaaggttctcaacgctggacaggtctgcatgtcacacaactacgtcctggttgacaaggatgttgctgacaccttcattgagttcctcaagatcgcatacaaggacatgttccctaacggtgccaaggcttcacctgatctctcacgtatcgtcaatgcacgtcacttcaaccgtatcaagaagatgctggatgagaccaagggtaagatcgtcatgggtggtgagatggacgagtctgagctgtacatcgaaccaactgctgtcctggttgactcactggacgaccctatgatgcaggaagaatccttcggacctatcttctccatctatcctgttgacactctggatcaggcactgtccatcgccaacaacgttcatcgtactccactggcactgatggcattcggtgacaagtctgagactaaccgtatcctggacgagatgacctctggtggtgcctgcatcaacgactcctactttcatggtgctgttcacaccgttccattcggtggtgttggtgattctggttggggtgcctaccgtggcaaagcctcattcgacaacttcactcacttccgtactgtgtctgagactccaacttggatggatcgtttccttcgtgttcgttacatgccatacgactggtctgaactgcgtcttcttcaacgttggaccaacaagaagcctaacttcgaccgtcaaggaaccgttgctaagggttctgagtactggatgtggtacttcctgggacttggcaccaagggtggtgtcaagggtgcactgatgcgttggcttgttgtcgttgctggctactacttgtctgcctacatgaaggcacgtcgtgcctaa

SEQ ID NO: 7 - DNA, 1374 bp, synthetic construct
  source 1..1374: mol_type = other DNA; note = An optimized nucleotide sequence for GjUGT74F8S gene
atgaactcatctaaggtgcatgtgttggttcttccttaccctgcacagggacatatcaacccacttcttcagttctgcaagagacttgtggctaagggtgtgaagactactttcgtgaacactgtgttcgtgtctaagtctatccagcctgatccttcatgctcaatcaacttcgagactatctctgatggacatgatgagggtggatacgctgctgctgagtcacctgaggcataccttgtggagcttgctactttcggatctaagactcttgctgatctcatcaggaagttggaggataagggtgagcctgtgcaggctgtgatctacgattcattcttgacttgggcattggatgtggctaagcagttcggacttgtgactgctgcattcttcactcagacttgcgctgtgaactctgtgtactatcatgtgtatcatggacttcttcctcttcctctctctgattcacctctctcattgcctggacttcctcttcttcagcctaaggagactccttcattcgtgtaccttcctgattcataccctgcattcagactcatgcttgtgaaccagttctctaacgtggatcaggcagattgggtgatcctcaacatcttccataagttggaggaggatgtgctcaactggatcgctaagctctggagagtgatcactgttggacctactgtgccatctatgtaccttgacaagagacttgaggatgacaagtgctacggtatcaacctcttcaagcctgattcatgcttgtgcatgaactggttgaacaaccagcctaaggattctgttgtgtacgtgtcattcggttcatggactgagatcgatgtgaagcaggtggaggagatcgcatcagcactcaaggagtctggtttcaagttcttgtgggttgtgagatcattcgagaaggagaagttgccttctaacttcgctgaggagacttctgagaagggacttgtggtgacttggtctcctcagcttgaggtgttggcacatgagtctgttgcttgcttcgtgactcattgcggtttcaactctgtgttggaggctctctcattgggtgttcctgttgtggctgctcctcaatggactgatcagcctactaacgctaagttcttggaggacatctggggtgttggtgtgaaggctggtgctgatgagaagggtatcgtgactagggagactcttgtgtcatgcatcagggagatcatggagggtgagaagggtaagcagatcaaggagaacgctatcaagtggaagactcttgctaaggaggctgtggatgagggtggatcttctgacaagaacatcgatgagttcgtggctgagttggctggacagaaggcacagaactga

SEQ ID NO: 8 - DNA, 1341 bp, synthetic construct
  source 1..1341: mol_type = other DNA; note = An optimized nucleotide sequence for UGT94E13S gene
atgaaggtgttgatgcttccttggttggcacatggacatatctctcctttcttggaacttgctaagaggttggctaagaagaacttccatatctacttgtgctctacttctgtgaatctctcatctatcaagaacaagatcactggtgagtactctgattctatcgagcctgtggagcttcagcttccttgcttgcctgatcttcctcctcattaccatactactaacggacttcctcctcatctcatgactactctcaagactgcatacgagttgtctgcacctgatttctctaacatcttgactgcattgcatcctgatcttgttgtgtacgatttcaatcagccttgggctgctgagatcgcatcatctaagaacatccctgctgtgcagttccttcctgtgggtgctactatgatggcattctctcttcacatgctcaagtactctggtaaggagttccctcatcctgaaatctacatcagagattacgagatgctcaagttccagtcaaggaacgatcaggtgaacgatgtgtctgacagagagaggatcttgcaggcacttgatctctcatgcaagattctcttggtgaagtcattcaaggagatcgaggagaagttcatgaacactctctctgttgcatctggtaagaaggtggttcctgtgggacctcttgtgcaggatgtgaacatcgatgacatccaggatgaggagatggagatcatccattggcttgatcagaaggagaacgcatctgtggtgttcgtgtctttcggttctgagtacttcttgactaaggaggagaggaacgagatcgctagaggacttgagttgtctaacgtgaacttcatctgggtgatcaggttccctcttggtggtaagatcactatggaggaggcacttcctgagggattccttgagagagtgggtgatagaggtaagatcgtggatggatgggcaccacaggctaggatcttgaaacatgctaacactggtgcattcttgtcacattgcggatggtcttcaatgatggagtctatgaagttcggtgtgcctatcatcgctatgcctatgtctgtggatcagcctgtgaacgctagattgatcgaggctgttggtgtgggattggagcctctcagagatgagaagggtaatcttcagtctgctgagatcgctaaggtgatcaggaaggtgttggtggatgagtctggtgagaacgtgaggaggaaggctaaggagttgtctgagcagatggagatgagaggtgatgaggaggagatcgacaaccttgtggaggagttgttgcagttgtgcaggaagaacaacggtggatgctga

SEQ ID NO: 9 - DNA, 258 bp, synthetic construct
  source 1..258: mol_type = other DNA; note = Nucleotide sequence for an expression promoter NRP33
ctttgctacccatcatgtatatatgatagccacaaagttactttgatgatgatatcaaagaacatttttaggtgcacctaacagaatatccaaataatatgactcacttagatcataatagagcatcaagtaaaactaacactctaaagcaaccgatgggaaagcatctataaatagacaagcacaatgaaaatcctcatcatccttcaccacaattcaaatattatagttgaagcatagtagtagaatccaacaaca

SEQ ID NO: 10 - DNA, 277 bp, synthetic construct
  source 1..277: mol_type = other DNA; note = An optimized nucleotide sequence for an Agrobacterium tumefaciens Nos terminator
agaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatc

SEQ ID NO: 11 - DNA, 1500 bp, synthetic construct
  source 1..1500: mol_type = other DNA; note = A nucleotide sequence of a marker gene SbG2S
atgcttggtgtgtctactcttcgttcaccttcacgttcatcttccaaggctgatcagcattgtggtggtggtttcgttggtgatcatcatgtggtgttcccaacttctggtggtggttctgactgctgcgatggtttcgcaatggttgatgttgacgacaacctgctggactacatcgacttctcatgtgatgttccattcttcgatgctgatggtgacatccttcctgacctggaggttgatccaactgaactgctggctgagttctcatcaactccaccacctgacgatctgcttcttgtgcatgttgatgatgaagcagccaacaacaaaccacctgtggtgcctgacgatgacgtgaagcagcatcatctggctgttgtcactactgctgctgctcaagaggagaagcagacctgtggtggtgacgagaagcatgtggctgcatccaagcagactactactgaggaggactactctgctgcatgtgcaggtgcagcagtgtctgataccaagtcatcagcatcagcagcagagtcacactccaagaagaaggctgctgctggtaacaagaactcacatggtaagcgtaaggtgaaggtggactggactcctgaactgcatcgtcgtttcgttcaggctgttgagcagcttggcatcgacaaggcagtgccatcacgtatcctggagatcatgggtatggactgtctgactcgtcacaacatcgcatcacaccttcagaagtaccgttcacaccgtaagcatctgatggcacgtgaagcagaagcagcaacttgggcacagaagcgtcacatgtatgcagcagctgctggtggcggtgcaccacgtactgatgcacctcacgcatcacgtccttgggttgttccaaccatcggtttcccaccacctgcaccaccaccattctgtcgtcctctgcatgtgtggggtcatccaccacatgcagctgctgctgaagcagcagctgctgtggcagcaccaccaactccaatgctgcctgtgtggccacgtcatctggcaccaccacgtcctctgccaccttgggcacatccacatccacctgctgttgatcctgcattctggcatcagcagtacaacgctgcacgtaagtggggtcctcaggctgctgctgtgactcaaggtactccatgtgttccaccacctgctgcaatgctgccacgtttccctgttcctggtatggttccacatccaatgtaccgtccaactatggttccaccaccaccaccaccacctccaccacagccatctactaagctggctgaactgcaactggagctggacactcatccatctaaggagtcaatcgatgctgcaatcggtgatgtgctggtgaagccttggcttccacttccacttggtctgaagccaccatcactggactctgtgatgtctgagctgcacaagcagggtgtgccatccatcccacctgctgcaactgcaactgcaatcgcaactggttaa

SEQ ID NO: 12 - DNA, 267 bp, synthetic construct
  source 1..267: mol_type = other DNA; note = An optimized nucleotide sequence encoding the Arabidopsis thaliana RbcS plastid transit peptide
atggcttcctctatgctctcttccgctactatggttgcctctccagctcaagccactatggttgccccattcaacggcctcaaatcctccgctgctttcccagccaccagaaaggctaacaacgacatcacttccatcacttccaacggcggaagagtcaactgtatgcaggtgtggcctccaatcggcaagaagaagttcgagactctctcttaccttcctgaccttaccgactccggtggcagagtcaactgtatgcaggccatg

SEQ ID NO: 13 - DNA, 21 bp, synthetic construct
  source 1..21: mol_type = other DNA; note = Nucleotide sequence for primer tHMG1S-F
ggtactaacgcacgtcaactg

SEQ ID NO: 14 - DNA, 21 bp, synthetic construct
  source 1..21: mol_type = other DNA; note = Nucleotide sequence for primer tHMG1S-R
ggatttgatgcaggtgacgga

SEQ ID NO: 15 - DNA, 23 bp, synthetic construct
  source 1..23: mol_type = other DNA; note = Nucleotide sequence for primer HpPSY1S-F
cagatcgctagagctagacagtg

SEQ ID NO: 16 - DNA, 23 bp, synthetic construct
  source 1..23: mol_type = other DNA; note = Nucleotide sequence for primer HpPSY1S-R
acctctgtgctgtggaaggagtg

SEQ ID NO: 17 - DNA, 24 bp, synthetic construct
  source 1..24: mol_type = other DNA; note = Nucleotide sequence for primer PscrtZS-F
tggtgcatgatggtctggttcatc

SEQ ID NO: 18 - DNA, 24 bp, synthetic construct
  source 1..24: mol_type = other DNA; note = Nucleotide sequence for primer PscrtZS-R
cttgccagatgaggaggtatccac

SEQ ID NO: 19 - DNA, 21 bp, synthetic construct
  source 1..21: mol_type = other DNA; note = Nucleotide sequence for primer PacrtIS-F
ttcgacttccgtgatcagctg

SEQ ID NO: 20 - DNA, 21 bp, synthetic construct
  source 1..21: mol_type = other DNA; note = Nucleotide sequence for primer PacrtIS-R
gatcaggtcctccagcatcag

SEQ ID NO: 21 - DNA, 24 bp, synthetic construct
  source 1..24: mol_type = other DNA; note = Nucleotide sequence for primer CsCCD2S-F
aggtacggctccgaggccatcttc

SEQ ID NO: 22 - DNA, 24 bp, synthetic construct
  source 1..24: mol_type = other DNA; note = Nucleotide sequence for primer CsCCD2S-R
ctcagcctggtgcttctgcaactc

SEQ ID NO: 23 - DNA, 24 bp, synthetic construct
  source 1..24: mol_type = other DNA; note = Nucleotide sequence for primer NcALD8S-F
gttcgttacatgccatacgactgg

SEQ ID NO: 24 - DNA, 23 bp, synthetic construct
  source 1..23: mol_type = other DNA; note = Nucleotide sequence for primer NcALD8S-R
acgacgtgccttcatgtaggcag

SEQ ID NO: 25 - DNA, 24 bp, synthetic construct
  source 1..24: mol_type = other DNA; note = Nucleotide sequence for primer GjUGT74F8S-F
tggctgctcctcaatggactgatc

SEQ ID NO: 26 - DNA, 24 bp, synthetic construct
  source 1..24: mol_type = other DNA; note = Nucleotide sequence for primer GjUGT74F8S-R
agccaactcagccacgaactcatc

SEQ ID NO: 27 - DNA, 24 bp, synthetic construct
  source 1..24: mol_type = other DNA; note = Nucleotide sequence for primer UGT94E13S-F
tgaacgctagattgatcgaggctg

SEQ ID NO: 28 - DNA, 24 bp, synthetic construct
  source 1..24: mol_type = other DNA; note = Nucleotide sequence for primer UGT94E13S-R
cagcatccaccgttgttcttcctg

(19) United States
(12) Patent Application Publication
(10) Pub. No.: US 2026/0250702 A1
(43) Pub. Date: Aug. 27, 2026

(54) GENE COMBINATION AND METHOD FOR BIOSYNTHESIS OF CROCINS

(71) Applicant: Shanghai Academy of Agricultural Sciences, Shanghai (CN)

(72) Inventors: Yongsheng TIAN, Shanghai (CN); Lijuan WANG, Shanghai (CN); Quanhong YAO, Shanghai (CN); Rihe PENG, Shanghai (CN); Bo WANG, Shanghai (CN); Jing XU, Shanghai (CN); Jianjie GAO, Shanghai (CN); Zhenjun LI, Shanghai (CN); Hongjuan HAN, Shanghai (CN); Xiaoyan FU, Shanghai (CN); Yongdong DENG, Shanghai (CN); Wenhui ZHANG, Shanghai (CN); Yu WANG, Shanghai (CN); Hao ZHANG, Shanghai (CN); Cen QIAN, Shanghai (CN)

(73) Assignee: Shanghai Academy of Agricultural Sciences, Shanghai (CN)

(21) Appl. No.: 19/093,323

(22) Filed: Mar. 28, 2025

(30) Foreign Application Priority Data
Feb. 24, 2025 (CN) 202510200381.6

(51) Int. Cl. C12N 15/82; C12N 9/02; C12N 9/04; C12N 9/10; C12N 9/90; C12N 15/52

(52) U.S. Cl. CPC C12N 15/8257; C12N 9/0006; C12N 9/0008; C12N 9/0077; C12N 9/1051; C12N 9/1085; C12N 9/90; C12N 15/52; C12N 15/8205; C12Y 101/01034; C12Y 102/01016; C12Y 114/15; C12Y 204/01276; C12Y 205/01001; C12Y 205/01032; C12Y 505/01019

(57) ABSTRACT

A gene combination for biosynthesis of crocins is provided. Each of eight genes, namely, tHMG1S gene, HpPSY1S gene, PscrtZS gene, PacrtIS gene, CsCCD2S gene, NcALD8S gene, GjUGT74F8S gene and UGT94E13S gene, obtained by codon optimization according to codon preference of rice is fused with a rice endosperm-specific phenotypic NRP33 promoter, a RbcS plastid transit peptide sequence and a Nos terminator to construct a gene expression cassette, all the expression cassettes are then ligated in series to construct a multigene plant vector for transformation into rice to obtain transgenic rice rich in crocins. The rice seeds are determined to have a crocin I content of 2.24 μg/g, a crocin II content of 1.65 μg/g and a crocin III content of 5.36 μg/g on dry weight basis. These rice seeds can be used as molecular farming for crocins production.

### CROSS REFERENCE TO THE RELATED APPLICATIONS

This application is based upon and claims priority to Chinese Patent Application No. 202510200381.6, filed on Feb. 24, 2025, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named WGJB0223_Sequence_Listing.xml, created on 03/27/2025, and is 44,327 bytes in size.

### TECHNICAL FIELD

The present disclosure belongs to the technical field of crop genetic engineering, and in particular relates to a gene combination for biosynthesis of crocins and a method for producing crocins by molecular farming.

### BACKGROUND

Crocins are main pharmacologically active compounds of the precious Chinese herbal material Croci stigma, which belongs to the class of water-soluble glycosylated carotenoids. Modern pharmacological studies have shown that crocins have anti-oxidant, anti-inflammatory, hypolipidemic, hypoglycemic, neuroprotective and anti-tumor properties. Clinical trials have shown that crocins play a positive role in the treatment of depression and Alzheimer's disease. Because of these excellent pharmacological functions, the market demand for crocins is increasing. However, market demand is far from being met due to resource constraints.

Currently, crocins are mainly extracted from expensive and rare Croci stigma, but the cost of manual picking greatly limits their large-scale production and widespread use. Moreover, due to the complex chemical structure and numerous chiral centers, crocins are difficult to obtain by chemical synthesis methods. Therefore, there is a need to seek alternative methods for sustainable crocins production.

### SUMMARY

An objective of the present disclosure is to provide a gene combination and method for biosynthesis of crocins, where the gene combination can be used to construct a multigene plant vector for transformation into a plant to obtain a transgenic plant rich in crocins.

In order to achieve the above objective, the present disclosure provides the following technical solutions:

The present disclosure provides a gene combination for biosynthesis of crocins, where the gene combination includes tHMG1S gene, HpPSY1S gene, PacrtIS gene, PscrtZS gene, CsCCD2S gene, NcALD8S gene, GjUGT74F8S gene and UGT94E13S gene, and nucleotide sequences of these genes are set forth in SEQ ID NOS: 1 to 8, respectively.

The tHMG1S gene, the HpPSY1S gene, the PacrtIS gene, the PscrtZS gene, the CsCCD2S gene, the NcALD8S gene, the GjUGT74F8S gene and the UGT94E13S gene are obtained by optimizing tHMG1 gene, HpPSY1 gene, PacrtI gene, PscrtZ gene, CsCCD2 gene, NcALD8 gene, GjUGT74F8 gene and UGT94E13 gene, respectively, on the basis of codon preference of rice.

The present disclosure further provides a multigene plant transformation vector, where the multigene plant transformation vector includes a plant expression vector and gene expression cassettes including the aforementioned gene combination, and the gene expression cassettes are constructed by fusing each of the tHMG1S gene, the HpPSY1S gene, the PscrtZS gene, the PacrtIS gene, the CsCCD2S gene, the NcALD8S gene, the GjUGT74F8S gene and the UGT94E13S gene with a NRP33 promoter and a Nos terminator, respectively.

As an embodiment, in the multigene plant transformation vector, the NRP33 promoter has the nucleotide sequence as set forth in SEQ ID NO: 9 and the Nos terminator has the nucleotide sequence as set forth in SEQ ID NO: 10.

As an embodiment, the multigene plant transformation vector further includes a gene expression cassette in which a marker gene SbG2S fused with a 35S promoter from cauliflower mosaic virus and the Nos terminator are fused, where the marker gene SbG2S has the nucleotide sequence as set forth in SEQ ID NO: 11.

In some embodiments, the PacrtIS gene, the PscrtZS gene, each of the CsCCD2S gene, the NcALD8S gene, the GjUGT74F8S gene and the UGT94E13S gene is further ligated to a sequence encoding an Arabidopsis thaliana RbcS plastid transit peptide, respectively. The sequence encoding the Arabidopsis thaliana RbcS plastid transit peptide is as set forth in SEQ ID NO: 12.

As an embodiment, the plant expression vector is a plant binary vector pYP694 obtained by introducing multiple cloning sites into pCamBIA-1301 as the backbone.

The present disclosure provides a method for biosynthesis of crocins, including: transforming the aforementioned multigene plant transformation vector into a plant to express crocins in the plant.

The present disclosure further provides a method for biosynthesis of crocins, where the specific operation steps are as follows:
1) sequentially cloning the expression cassettes of the eight genes tHMG1S, HpPSY1S, PscrtZS, PacrtIS, CsCCD2S, NcALD8S, GjUGT74F8S and UGT94E13S and the SbG2S gene expression cassette into the plant expression vector pYP694 to obtain a recombinant plasmid vector pYR48;
2) transforming the recombinant plasmid vector pYR48 into a host strain;
3) infecting calli with the host strain, and screening resistant calli;
4) differentiation culturing the resistant calli to obtain a transgenic plant expressing crocins.

As an embodiment, the plant is a gramineous plant or a leguminous plant.

In some embodiments, the host strain in step 2) is Agrobacterium tumefaciens EHA105.

In some embodiments, the calli in step 3) are selected from gramineous plants or leguminous plants, including rice, wheat, maize, sorghum, millet and soybean.

Beneficial effects: In the present disclosure, the tHMG1 gene, the HpPSY1 gene, the HpcrtZ gene, the PacrtI gene, the CsCCD2 gene, the NcALD8 gene, the GjUGT74F8 gene and the UGT94E13 gene combination are optimized, and the optimized genes are expressed in a plant to achieve the biosynthesis of crocins. The resulting transgenic rice has a crocin I content of 2.24 μg/g, a crocin II content of 1.65 μg/g and a crocin III content of 5.36 μg/g in the endosperm. The transgenic rice germplasm can be used as molecular farming, i.e., a raw material, for the production of crocins, and can also be used as health-promoting functional rice.

### BRIEF DESCRIPTION OF THE DRAWINGS

FIG. 1 shows a schematic diagram of the constructed vector for biosynthesis of crocins in rice endosperm;

FIG. 2 shows the phenotypes of green calli of rice;

FIG. 3 shows the expression of crocin biosynthetic genes in rice, as detected by PCR; M: marker; CK: wild-type rice; EH887-2, 3, 4: i.e., 887-2, 887-3, and 887-4, which are different rice lines created in the present disclosure;

FIG. 4 shows the color of rice seeds; top three rows: the color of rice seeds created in the present disclosure; and the last row: the color of wild-type rice seeds;

FIGS. 5A-5B show the content of crocins in rice seeds, as detected by LC/MS, where FIG. 5A, from left to right, shows the mass spectrum of crocin standards, crocin I standard, crocin II standard and crocin III standard, respectively, from left to right, and FIG. 5B, from left to right, shows the mass spectrum of crocins in rice seeds created in the present disclosure, crocin I, crocin II and crocin III, respectively.

### DETAILED DESCRIPTION OF THE EMBODIMENTS

The present disclosure provides a gene combination for biosynthesis of crocins, where the gene combination includes tHMG1S gene, HpPSY1S gene, PacrtIS gene, PscrtZS gene, CsCCD2S gene, NcALD8S gene, GjUGT74F8S gene and UGT94E13S gene.

The above genes are obtained by optimizing tHMG1 gene, HpPSY1 gene, PscrtZ gene, PacrtI gene, CsCCD2 gene, NcALD8 gene, GjUGT74F8 gene and UGT94E13 gene, respectively, according to codon preferences of rice. The optimization principles are as follows: 1. optimizing gene codons according to codon preferences of rice to increase gene translation efficiency; 2. eliminating recognition sites for common restriction endonucleases within the gene to facilitate the construction of expression cassettes; 3. eliminating inverted repeat sequences, stem-loop structures and transcription termination signals to balance the GC/AT ratio within the gene, thereby improving RNA stability, where the GC/AT ratio within the gene is as far as possible to achieve 1:1; 4. allowing the protein encoded by the gene to conform to the N-end rule to improve the stability of the translated protein; 5. optimizing the free energy of mRNA secondary structure to improve gene expression efficiency. When optimizing the secondary structure, sequences containing six or more consecutive A+T are avoided, and sequences containing five or more G+C are avoided; the case where dinucleotides CG or TA are located at positions 2 and 3 is avoided; in addition, as much as possible, the free energy ΔG at the 5′ end of the gene is increased by 5.4 Kcal or more, and the free energy at the 3′ end is reduced to 10.8 Kcal or less. By means of the above optimization principles, the optimized genes can be efficiently expressed in the rice seed endosperm. Finally the eight optimized genes, namely, tHMG1S, HpPSY1S, PacrtIS, PscrtZS, CsCCD2S, NcALD8S, GjUGT74F8S and UGT94E13S, are obtained, the nucleotide sequences of these eight genes are as set forth in SEQ ID NOS: 1 to 8.

The present disclosure further provides a multigene plant transformation vector, where the multigene plant transformation vector includes a plant expression vector and a gene expression cassette containing tHMG1S gene, HpPSY1S gene, PscrtZS gene, PacrtIS gene, CsCCD2S gene, NcALD8S gene, GjUGT74F8S gene and UGT94E13S gene. The gene expression cassettes involve expression units fused by ligating a rice endosperm-specific phenotypic promoter NRP33 promoter and a Nos terminator at both ends of each of the eight genes described above, respectively, where the NRP33 promoter has the nucleotide sequence as set forth in SEQ ID NO: 9 and the Nos terminator has a nucleotide sequence as set forth in SEQ ID NO: 10. The PacrtIS gene, the PscrtZS gene, the CsCCD2S gene, the NcALD8S gene, the GjUGT74F8S gene and the UGT94E13S gene are further ligated to a sequence encoding an Arabidopsis thaliana RbcS plastid transit peptide, respectively. The sequence encoding the Arabidopsis thaliana RbcS plastid transit peptide is as set forth in SEQ ID NO: 12. The multigene plant transformation vector of the present disclosure further includes a gene expression cassette of a marker gene SbG2S for green callus formation fused with a 35S promoter from cauliflower mosaic virus and the Nos terminator, where the marker gene SbG2S has a nucleotide sequence as set forth in SEQ ID NO: 11. For the plant expression vector of the present disclosure, pCamBIA-1301 can be selected, or new multiple cloning sites can be introduced into pCamBIA-1301 as the backbone to modify the vector to obtain a plant binary vector pYP694. Preferably, a plant binary vector pYP694 can be selected as the plant expression vector. The expression units of the eight optimized genes described above together with the marker gene SbG2S are sequentially cloned into the plant expression vector to obtain a recombinant plasmid vector. The cloning method refers to the patent CN 114592000 B.

The present disclosure further provides a method for biosynthesis of crocins, including: transforming the multigene plant transformation vector of the present disclosure into a plant to express the crocins in the plant. As an implementation, the specific operation steps are: 1) sequentially cloning the expression cassettes of the eight genes tHMG1S, HpPSY1S, PscrtZS, PacrtIS, CsCCD2S, NcALD8S, GjUGT74F8S and UGT94E13S and the SbG2S gene expression cassette into the plant expression vector pYP694 to obtain a recombinant plasmid vector pYR48; 2) transforming the recombinant plasmid vector pYR48 into a host strain; 3) infecting calli with the host strain, and screening resistant calli; 4) differentiation culturing the resistant calli to obtain a crocin-expressing transgenic plant. As an embodiment, the host strain in step 2) is Agrobacterium tumefaciens EHA105. As an embodiment, the calli in step 3) may be selected from gramineous plants, including rice, wheat, maize, sorghum, millet, etc., or may also be selected from leguminous plants, including soybean, etc. Preferably, the calli are selected from rice embryogenic calli. The method for determining the content of crocins in rice of the present disclosure refers to the document: Mi, Jianing et al. “A rapid LC-MS method for qualitative and quantitative profiling of plant apocarotenoids.” Analytica chimica acta vol. 1035 (2018): 87-95. doi:10.1016/j.aca.2018.07.002.

In the present disclosure, unless otherwise specified, all raw material components are commercially available and well known to those skilled in the art. The present disclosure relates to experiments in molecular biology, all of which refer to the book “Molecular Cloning: A Laboratory Mannual” (J. Sambrook, E. F. Fritsch, and T. Maniatis, 1994, Science Press). The rice seeds used (japonica variety Nipponbare) are preserved in the Research Center for Agricultural Synthetic Biology, The Biotechnology Research Center of Shanghai Academy of Agriculture Sciences. Unless specified, the reagents used in the present disclosure are purchased from Sangon Biotech (Shanghai) Co., Ltd. or China National Pharmaceutical Group Co., Ltd. Unless specified, the culture media used in the present disclosure may refer to the patent CN 114592000 A.

The technical solutions in the present disclosure will be described clearly and completely below with reference to the examples of the present disclosure. Apparently, the described examples are merely a part of, rather than all of the examples of the present disclosure. Based on the examples of the present disclosure, all other examples that can be obtained by those of ordinary skill in the art without creative efforts shall fall within the scope of protection of the present disclosure.

### Example 1

### Structural Optimization and Chemical Synthesis of Eight Genes Involved in Crocin Synthesis Pathway

The eight genes for optimized synthesis are: tHMG1 gene (GenBank No: M22002.1, 1771 . . . 3285), HpPSY1 gene (GenBank No: DQ057355.1), PacrtI gene (GenBank No: D90087.2), PscrtZ gene (GenBank No: KP866868.1), CsCCD2 gene (GenBank No: KJ541749.1), NcALD8 gene (GenBank No: XM_011396597.1), GjUGT74F8 gene (GenBank No: MN944054.1), and UGT94E13 gene (GenBankNo: KY631935.1).

The eight optimized and synthesized genes are named: tHMG1S gene, HpPSY1S gene, PacrtIS gene, PscrtZS gene, CsCCD2S gene, NcALD8S gene, GjUGT74F8S gene and UGT94E13S gene, respectively, and their sequence ID numbers correspond to SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8, respectively.

### Example 2

### Construction of Gene Expression Cassette

A rice (Oryza sativa) endosperm-specific expression promoter NRP33 having the DNA sequence as set forth in SEQ ID NO: 9 was synthesized using the sequence of Gene Bank No: D63901.1 as a template.

An Agrobacterium tumefaciens Nos terminator having the DNA sequence as set forth in SEQ ID NO: 10 was synthesized using the sequence of GenBank No: AB830573.1 as a template.

A sequence encoding the plastid transit peptide RbcS TP was optimized with reference to the sequence of Arabidopsis thaliana RbcS small subunit gene (Gene Bank No: NM_105379.4) and synthesized (SEQ ID NO: 12).

The SbG2S gene having the DNA sequence as set forth in SEQ ID NO: 11 that can promote chloroplast development was optimized and synthesized using the sequence of GenBank No: XM_021456623.1 as a template.

The fusion of the gene with the plastid transit peptide, the promoter and the terminator was performed using a modified overlap extension PCR technique, which was described specifically in the references: (Rihe Peng et al., Applied Microbiology Biotechnology. 2006, 73:234-240 and ZL 2020 1 1411342.4). In the order of NRP33 promoter, tHMG1S gene and Nos terminator, the sequences of the three were fused to construct the NRP33tHMG1S gene expression cassette. In the order of NRP33 promoter, HpPSY1S gene and Nos terminator, the sequences of the three were fused to construct the NRP33HpPSY1S gene expression cassette. In the order of NRP33 promoter, RbcS, PacrtIS gene and Nos terminator, the sequences of the four were fused to construct the NRP33RPacrtIS gene expression cassette. In the order of NRP33 promoter, RbcS, PscrtZS gene and Nos terminator, the sequences of the four were fused to construct the NRP33RPscrtZS gene expression cassette. In the order of NRP33 promoter, RbcS, CsCCD2S gene and Nos terminator, the sequences of the four were fused to construct the NRP33R3CsCCD2S gene expression cassette. In the order of NRP33 promoter, RbcS, NcALD8S gene and Nos terminator, the sequences of the four were fused to construct the NRP33RNcALD8S gene expression cassette. In the order of NRP33 promoter, RbcS, GjUGT74F8S gene and Nos terminator, the sequences of the four were fused to construct the NRP33RGjUGT74F8S gene expression cassette. In the order of NRP33 promoter, RbcS, UGT94E13S gene and Nos terminator, the sequences of the four were fused to construct the NRP33RUGT94E13S gene expression cassette. In the order of 35S promoter, SbG2S gene and Nos terminator, the sequences of the three were fused to construct the 35S SbG2S gene expression cassette.

PCR amplification was performed using Phanta Max Super-Fidelity DNA Polymerase from Vazyme Biotech Co., Ltd that is suitable for high-fidelity amplification of long genes. The PCR amplification program was: pre-denaturation at 95° C. for 30 s; denaturation at 95° C. for 45 s, annealing at 56-72° C. for 45 s, extension at 72° C. for 5-20 min (depending on fragment length), 25-35 cycles; final extension at 72° C. for 10 min. The complete sequence was subjected to full sequence analysis by Sangon Biotech (Shanghai) Co., Ltd.

### Example 3

### Construction of Plant Expression Vector

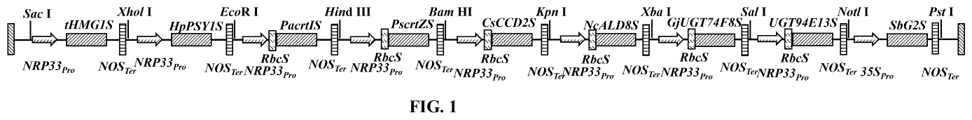

The nine gene expression cassettes constructed in Example 2 that were sequenced correctly were ligated into the pYP694 vector according to the method of patent CN 114592000 B to obtain a recombinant plant expression vector pYR48 containing a total of nine genes, i.e., the genes involved in the crocin synthesis pathway and a marker gene, as shown in FIG. 1, where the pYP694 vector referred to Tian Y S et al. Enhancing carotenoid biosynthesis in rice endosperm by metabolic engineering. Plant Biotechnol J. 2019 May; 17(5): 849-851.

### Example 4

### Rice Transformation

1) Preparation of recombinant Agrobacterium

The single colonies of Agrobacterium tumefaciens EHA105 were selected and inoculated into 5 mL of LB liquid medium (rifampicin 50 μg/mL, chloramphenicol 100 μg/mL), and cultured at 28° C., 250 rpm for 20 h. 1 mL of the bacterial solution was transferred and inoculated into 20 mL of LB liquid medium (rifampicin 50 μg/mL, chloramphenicol 100 μg/mL), and cultured at 28° C., 250 rpm for about 12 h, with the measured OD₆₀₀ of about 1.5. The bacteria were collected by centrifugation at 8000 rpm, 4° C. for 10 min, then resuspended in Agrobacterium transformation permeate (5 wt % sucrose, 0.05 wt % Silwet L-77), and diluted to an OD₆₀₀ of about 0.8. 40 μl of the bacterial solution and 2 μl of the recombinant plant expression vector pYR48 were uniformly mixed in an electroporation cuvette with a diameter of 0.2 cm, and placed on ice for 1 min. The electroporation parameters were adjusted to 25 μF, 2.5 kV/cm, 400Ω, and the discharge time was 4-5 ms. After electroporation, 1 ml of LB medium was added immediately into the bacteria, cultured at 28° C. for 1 h, and the bacteria were coated on YEB plates (rifampicin 50 μg/mL, kanamycin 100 μg/mL). Using the above transformation procedures, the above expression vector pYR48 was transformed into Agrobacterium tumefaciens EHA105 to obtain Agrobacterium tumefaciens EHA105 (pYR48).

2) Agrobacterium Infection and Co-Culture with Rice Calli

Rice embryogenic calli were immersed in the prepared Agrobacterium suspension. After 30 min of infection, the calli were placed on sterile filter paper to remove the excess bacterial solution by absorbing, and transferred into a culture medium N6 (containing 2,4-D 1.5 mg/L) and co-cultured at 28° C. in the dark for 3-4 d.

### 3) Screening of Resistant Calli

The co-cultured calli were transferred out, rinsed with sterile water 3-4 times, and placed on sterile filter paper to remove the excess water. The resulting calli were transferred into selective medium N6 (containing 2,4-D 1.5 mg/L, hygromycin 25 mg/L), and cultured at 28° C. in the dark, with subculturing performed every two weeks.

### 4) Plant Regeneration

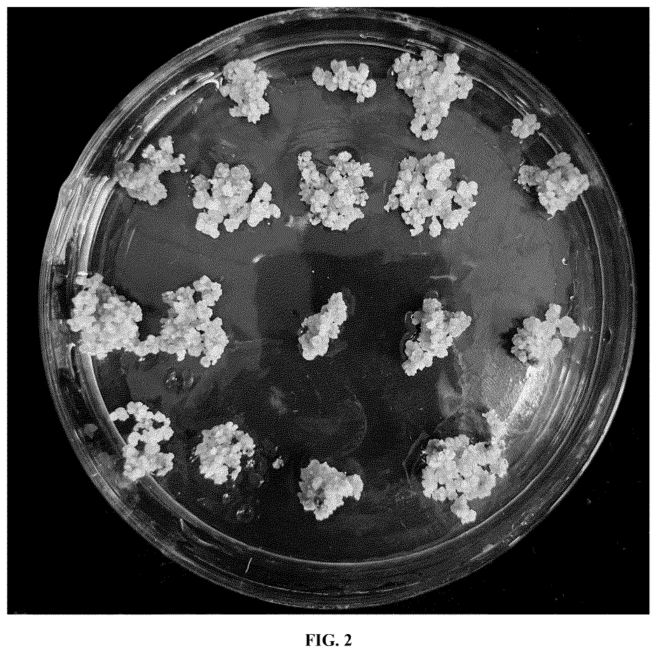

After 2-3 passages of screening, vigorous green calli were selected, as shown in FIG. 2, and transferred into pre-differentiation medium MS (containing 6-BA 1 mg/L, KT 1 mg/L, IAA 0.05 mg/L, hygromycin 25 mg/L) for pre-differentiation treatment: after 5-7 days of culture in the dark, the resistant calli were then transferred into differentiation medium MS (containing 6-BA 4 mg/L, KT 4 mg/L, IAA 0.2 g/L, hygromycin 25 mg/L), and differentiation culturing was performed under conditions of 16 h light and 8 h darkness daily at 28° C. The regenerated seedlings were trimmed of their original roots and subjected to rooting and seedling strengthening in rooting medium MS, then transferred into pots in a controlled climate chamber to obtain transgenic rice, where the humidity was maintained for the first few days, and the subsequent cultivation management was performed in accordance with conventional methods.

### Example 5

### Verification of Transgenic Rice

For the seeds harvested from the T₀-generation rice plants obtained in Example 4, the genomic DNA was extracted by SDS method as a template and foreign genes were detected by PCR. The sequences of the designed primers were as follows:

tHMG1S-F:
(SEQ ID NO: 13)
5′-GGTACTAACGCACGTCAACTG;
tHMG1S-R:
(SEQ ID NO: 14)
5′-GGATTTGATGCAGGTGACGGA.
HpPSY1S-F:
(SEQ ID NO: 15)
5′-CAGATCGCTAGAGCTAGACAGTG;
HpPSY1S-R:
(SEQ ID NO: 16)
5′-ACCTCTGTGCTGTGGAAGGAGTG.
PscrtZS-F:
(SEQ ID NO: 17)
5′-TGGTGCATGATGGTCTGGTTCATC;
PscrtZS-R:
(SEQ ID NO: 18)
5′-CTTGCCAGATGAGGAGGTATCCAC.
PacrtIS-F:
(SEQ ID NO: 19)
5′-TTCGACTTCCGTGATCAGCTG;
PacrtIS-R:
(SEQ ID NO: 20)
5′-GATCAGGTCCTCCAGCATCAG.
CsCCD2S-F:
(SEQ ID NO: 21)
5′-AGGTACGGCTCCGAGGCCATCTTC;
CsCCD2S-R:
(SEQ ID NO: 22)
5′-CTCAGCCTGGTGCTTCTGCAACTC.
NcALD8S-F:
(SEQ ID NO: 23)
5′-GTTCGTTACATGCCATACGACTGG;
NcALD8S-R:
(SEQ ID NO: 24)
5′-ACGACGTGCCTTCATGTAGGCAG.
GjUGT74F8S-F:
(SEQ ID NO: 25)
5′-TGGCTGCTCCTCAATGGACTGATC;
GjUGT74F8S-R:
(SEQ ID NO: 26)
5′-AGCCAACTCAGCCACGAACTCATC.
UGT94E13S-F:
(SEQ ID NO: 27)
5′-TGAACGCTAGATTGATCGAGGCTG;
UGT94E13S-R:
(SEQ ID NO: 28)
5′-CAGCATCCACCGTTGTTCTTCCTG.

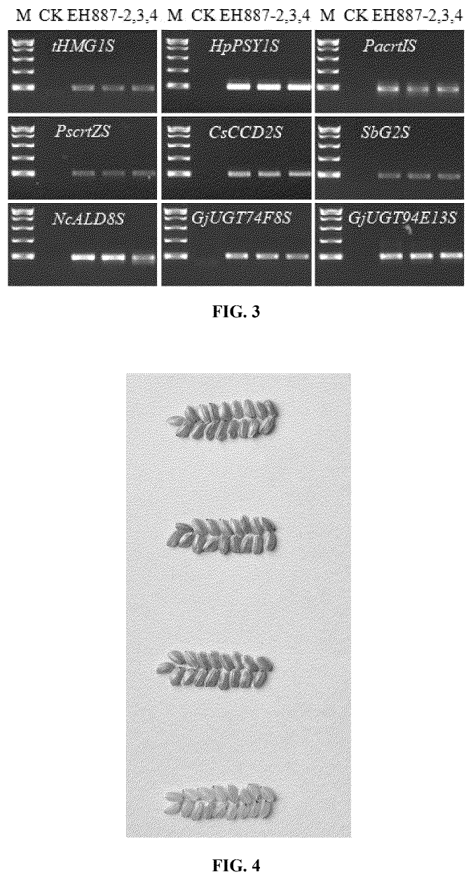

Amplification program: 94° C. 30 s, 54° C. 30 s, 72° C. 30 s, a total of 45 cycles, and final extension at 72° C. for 10 min. As shown in FIG. 3, the results show that no foreign gene was amplified in the wild-type (CK), while all eight genes described above were amplified in all the transgenic lines, indicating that all foreign genes were completely integrated into the rice genome.

### Example 6

### Determination on the Content of Crocins

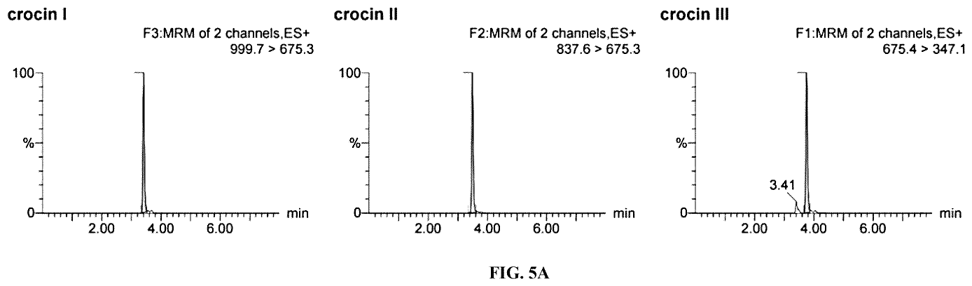

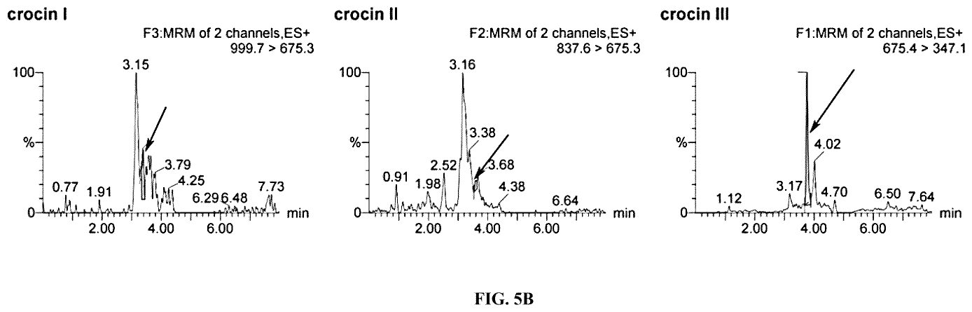

The rice plants detected positive in Example 5 were planted into the farmland according to the following steps:
1) Suitable land was selected, plowed, fertilized, irrigated, etc. to ensure the fertility and moisture of the soil to meet the needs of rice growth.
2) The selected rice seeds were sown in the field, where there were two sowing methods: direct sowing and seedling nursery, the direct sowing involved directly scattering seeds in the field, and the seedling nursery involved first planting seedlings in the seedling field, and then transplanting the seedlings to the field once they grew to a certain height.
3) After sowing, regular field management was required, including weeding, irrigation, fertilization, control of disease and pest, etc.
4) The rice was harvested after maturation, where the harvest could be performed manually or by machine.
5) The harvested rice grains were threshed, and it was clearly observed that the seeds exhibited a yellow color (see FIG. 4), indicating the biosynthesis of crocins in the rice seeds.
6) The seeds exhibiting the yellow color were subjected again to steps 2) to 5) until homozygous seeds were obtained.
7) The homozygous seeds of different lines were dehulled. 1-1.5 g of the dehulled homozygous seeds were weighed and crushed. About 0.5 g of the resulting powder was taken, 1 mL of 50% aqueous methanol solution was added as an extraction solution, and steel beads were added for homogenization. Ultrasonic extraction was performed for 60 min. Centrifugation was performed at 8000 g for 10 min. The supernatant was taken, filtered using a syringe filter, and subjected to qualitative and quantitative analysis by LC/MS, where the analysis method was described in the references (J. Mi et al. (2018) Analytica Chimica Acta 1035, 87-95.). The rice line seeds with the highest crocins content were determined as below: a crocin I content of 2.24 μg/g, a crocin II content of 1.65 μg/g, and a crocin III content of 5.36 μg/g (dry weight), as shown in FIGS. 5A-5B.

Although the examples described above have provided a detailed description of the present disclosure, they are only some, rather than all of the examples of the present disclosure. All other examples that can be obtained according to the examples of the present disclosure without involving any inventive effort shall fall within the scope of protection of the present disclosure.

What is claimed is:

1. A multigene plant transformation vector, comprising a plant expression vector and gene expression cassettes of a gene combination, wherein
the gene combination comprises a tHMG1S gene, a HpPSY1S gene, a PacrtIS gene, a PscrtZS gene, a CsCCD2S gene, a NcALD8S gene, a GjUGT74F8S gene, and a UGT94E13S gene having the nucleotide sequences as set forth in SEQ ID NOS: 1 to 8, respectively; wherein
the tHMG1S gene, the HpPSY1S gene, the PacrtIS gene, the PscrtZS gene, the CsCCD2S gene, the NcALD8S gene, the GjUGT74F8S gene and the UGT94E13S gene are obtained by optimizing a tHMG1 gene, a HpPSY1 gene, a PacrtI gene, a PscrtZ gene, a CsCCD2 gene, a NcALD8 gene, a GjUGT74F8 gene and a UGT94E13 gene, respectively, according to codon preference of rice; and
the gene expression cassettes are constructed by fusing the tHMG1S gene, the HpPSY1S gene, the PscrtZS gene, the PacrtIS gene, the CsCCD2S gene, the NcALD8S gene, the GjUGT74F8S gene and the UGT94E13S gene with a NRP33 promoter and a Nos terminator, respectively.

2. The multigene plant transformation vector of claim 1, wherein the NRP33 promoter has the nucleotide sequence as set forth in SEQ ID NO: 9 and the Nos terminator has the nucleotide sequence as set forth in SEQ ID NO: 10.

3. The multigene plant transformation vector of claim 1, further comprising a gene expression cassette of a marker gene SbG2S, wherein the marker gene SbG2S is fused with a 35S promoter from cauliflower mosaic virus and the Nos terminator; and the marker gene SbG2S has the nucleotide sequence as set forth in SEQ ID NO: 11.

4. The multigene plant transformation vector of claim 1, wherein the PacrtIS gene, the PscrtZS gene, the CsCCD2S gene, the NcALD8S gene, the GjUGT74F8S gene and the UGT94E13S gene are further ligated to a sequence encoding an Arabidopsis thaliana RbcS plastid transit peptide, respectively; and the sequence encoding the Arabidopsis thaliana RbcS plastid transit peptide is as set forth in SEQ ID NO: 12.

5. The multigene plant transformation vector of claim 1, wherein the plant expression vector is a plant binary vector pYP694 obtained by introducing multiple cloning sites into pCamBIA-1301 as a backbone.

6. A method for a biosynthesis of crocins, comprising transforming the multigene plant transformation vector of claim 1 into a plant to express the crocins in the plant.

7. The method for the biosynthesis of the crocins of claim 6, wherein the method comprises:
1) sequentially cloning the gene expression cassettes and a SbG2S gene expression cassette into a plant expression vector pYP694 to obtain a recombinant plasmid vector pYR48;
2) transforming the recombinant plasmid vector pYR48 into a host strain;
3) infecting calli with the host strain, and screening resistant calli; and
4) differentiation culturing the resistant calli to obtain a transgenic plant expressing the crocins.

8. The method for the biosynthesis of the crocins of claim 6, wherein the plant is a gramineous plant or a leguminous plant.

9. The method for the biosynthesis of the crocins of claim 7, wherein the host strain in the step 2) is Agrobacterium tumefaciens EHA105.

10. The method for the biosynthesis of the crocins of claim 7, wherein the calli in the step 3) are gramineous plants or leguminous plants.

11. The method for the biosynthesis of the crocins of claim 10, wherein the calli in the step 3) are one selected from the group consisting of the rice, wheat, maize, sorghum, millet, and soybean.
